(19) United States
(12) Patent Application Publication
(10) Pub. No.: US 2022/0373885 A1
(43) Pub. Date: Nov. 24, 2022

(54) BISPHENOL A DERIVATIVE, PREPARATION METHOD THEREFOR AND USE THEREOF IN PHOTOLITHOGRAPHY

(71) Applicant: INSTITUTE OF CHEMISTRY, CHINESE ACADEMY OF SCIENCES, Beijing (CN)

(72) Inventors: Guoqiang YANG, Beijing (CN); Yafei WANG, Beijing (CN); Long CHEN, Beijing (CN); Jiating YU, Beijing (CN); Rui HU, Beijing (CN); Xudong GUO, Beijing (CN); Shuangqing WANG, Beijing (CN)

(21) Appl. No.: 17/753,898

(22) PCT Filed: Dec. 10, 2020
(86) PCT No.: PCT/CN2020/135313

§ 371 Date: Mar. 17, 2022

(30) Foreign Application Priority Data
Aug. 11, 2020 (CN) 202010803879.9

(51) Int. Cl. G03F 7/004; G03F 7/038; C07D 301/28; C07D 303/27

(52) U.S. Cl. CPC G03F 7/0045; G03F 7/038; C07D 301/28; C07D 303/27

(57) ABSTRACT

A bisphenol A derivative, a preparation method therefor and use thereof in photolithography are provided. The compounds feature simple molecular structure, controllable molecular weight, simple synthesis steps, and relatively high thermal stability. They do not precipitate during baking and are not easily denatured during photolithography. The negative molecular glass photoresists have good film-forming property, high thermal stability, less proneness to properties varying during storage, and low viscosity, no need for additional solvents for dilution during use. After exposure at UV wavelength of 365 nm, the exposed pattern shows high contrast, excellent resolution and good sensitivity, and can present the lithographic line width of 3.5 μm.

The present invention claims priority to Chinese Patent Application No. 202010803879.9 filed with China National Intellectual Property Administration on Aug. 11, 2020 and entitled “BISPHENOL A DERIVATIVE, PREPARATION METHOD THEREFOR AND USE THEREOF IN PHOTOLITHOGRAPHY”, the content of which is incorporated herein by reference in its entirety.

### TECHNICAL FIELD

The present invention belongs to the technical field of photolithography, and particularly relates to a bisphenol A derivative, a preparation method therefor and use thereof in photolithography.

### BACKGROUND

With the emergence of the integrated circuit, especially the very large scale integrated circuit, the microelectronic technology has been developed, making it one of the core technologies in the information industry, which has a profound influence on national economy. In the late 1950s, scientists invented germanium integrated circuits and silicon integrated circuits. The emergence of integrated circuits has driven the rapid development of semiconductor technology. Modern electronic devices require integrated circuits (chips) with increasingly smaller dimensions and higher integration levels. Since the 1980s, the photolithography technique has been developed from G-line (436 nm) and I-line (365 nm) lithography, to deep ultraviolet (248 nm and 193 nm) lithography, and then to the next generation of lithography techniques such as extreme ultraviolet lithography (EUVL), nanoimprint lithography, and electron beam lithography, and the corresponding photoresists have also changed. Advanced photolithography techniques have enabled increasingly higher integration levels and smaller dimensions. The minimum feature dimension of integrated circuits has moved from the micron scale and the submicron scale to the nanometer scale.

Photolithography techniques and related processes thereof are the mainstay of the nanotechnology revolution, and the feature dimensions of microelectronic circuit elements fabricated by current optical photolithography processes are limited by the wavelength of the exposure radiation. Researchers have been developing novel photolithography techniques and photoresist materials to meet the ever-increasing demand for higher resolution and better sensitivity. Extreme Ultraviolet (EUV) lithography and electron beam lithography are capable of producing nanoscale patterns with minimum feature dimensions below 10 nm. In the development of semiconductor devices with faster speeds and smaller dimensions, novel photoresist materials and photolithography processes are currently being explored jointly by industrial and academic communities.

With the shift in exposure wavelength from ultraviolet (UV) to deep ultraviolet (DUV) to extreme ultraviolet (EUV), researchers have been developing more photoresist systems, with chemically amplified resists (CARs) dominating the semiconductor industry manufacturing. In conventional CARs, there is an insurmountable interplay between resolution, line edge roughness and sensitivity (RLS) for a given photoresist material/formulation, that is, it is not possible to optimize these three parameters simultaneously. Any two of these three parameters may be improved, but only at the cost of a reduced performance of the third parameter. One of the main reasons why CARs have difficulty meeting these three parameters simultaneously is the diffusion of the photoacid generator. Increasing the diffusion of the photoacid generator improves the sensitivity of CARs but results in reduced resolution and more complex effects on line edge roughness (LER). Reduced diffusion of the photoacid generator improves the resolution but results in loss of sensitivity, with a possible increase of LER.

### SUMMARY

One object of the present invention is to provide a bisphenol A derivative (I) and a preparation method therefor.

Another object of the present invention is to provide a negative photoresist composition comprising the bisphenol A derivative (I).

The present invention provides a compound of formula (I):

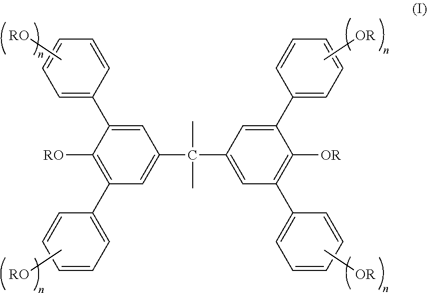

wherein R is H,

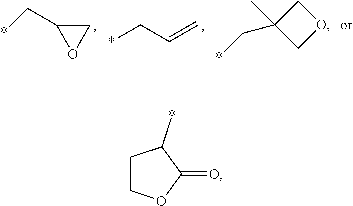

provided that R is not all H, the * in the above groups is a linking site; n is 1, 2, 3, 4 or 5, preferably 1, 2 or 3.

According to an embodiment of the present invention, the compound of formula (I) is selected from a compound of formula (IA) and a compound of formula (IB) below,

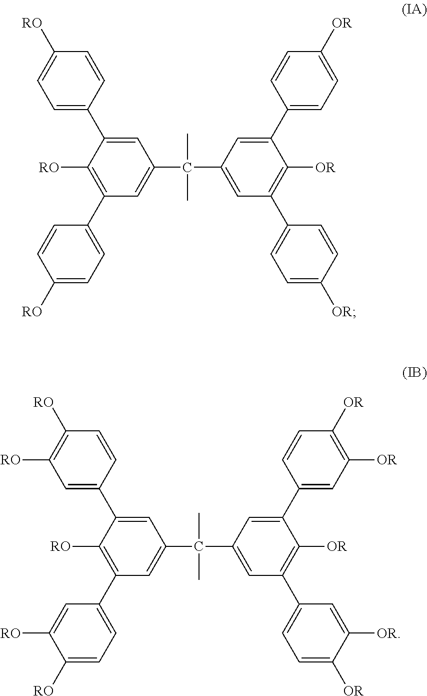

According to an embodiment of the present invention, R is

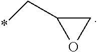 [SMILES: *CC1CO1]

According to an embodiment of the present invention, the compound of formula (I) is selected from the following compounds:

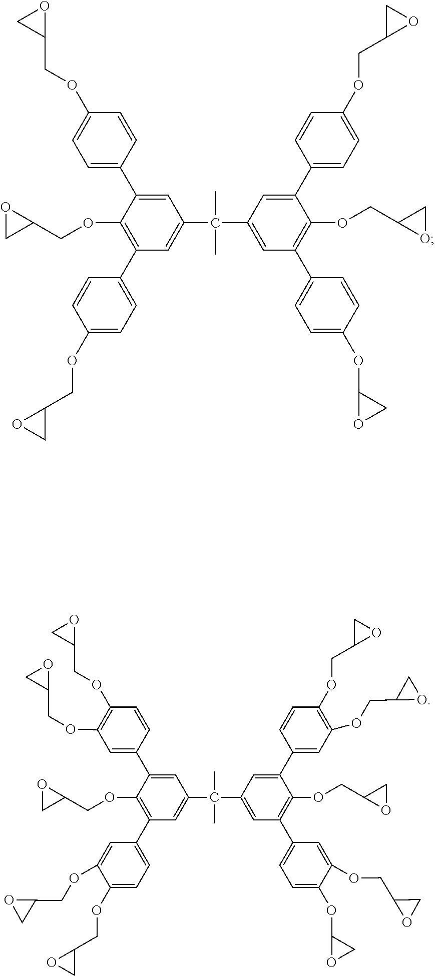 [SMILES: CC(C)(c1cc(-c2ccc(OCC3CO3)cc2)c(OCC2CO2)c(-c2ccc(OCC3CO3)cc2)c1)c1cc(-c2ccc(OCC3CO3)cc2)c(OCC2CO2)c(-c2ccc(OC3CO3)cc2)c1 ; CC(C)(c1cc(-c2ccc(OCC3CO3)c(OCC3CO3)c2)c(OCC2CO2)c(-c2ccc(OCC3CO3)c(OCC3CO3)c2)c1)c1cc(-c2ccc(OCC3CO3)c(OCC3CO3)c2)c(OCC2CO2)c(-c2ccc(OC3CO3)c(OCC3CO3)c2)c1]

The present invention further provides a method for synthesizing the compound of formula (I) as described above, comprising:

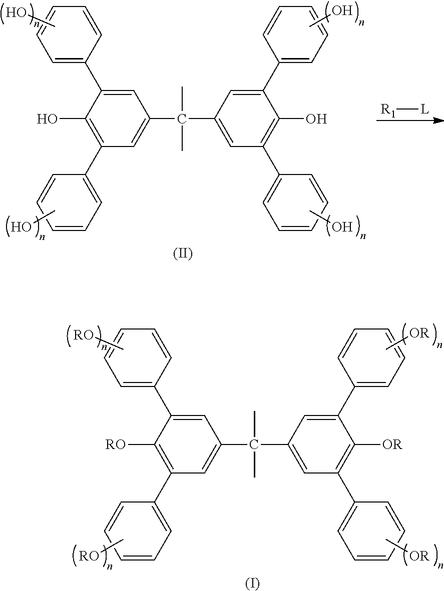

reacting a compound of formula (II) with R₁-L to give the compound of formula (I);
wherein R and n are as defined above, R₁ is

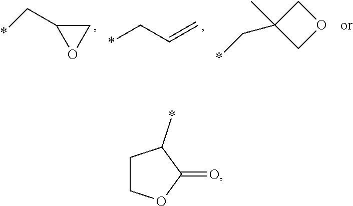

and L is selected from a leaving group such as halogen or p-toluenesulfonate.

According to an embodiment of the present invention, R₁-L is selected from epibromhydrin, allyl bromide, α-bromo-γ-butyrolactone and 3-methyl-3-(tosyloxymethyl)oxetane.

The compound (I) described herein is obtained by introducing R₁ groups on the compound of formula (II) for complete or partial protection.

According to an embodiment of the present invention, L is selected from bromine.

According to an embodiment of the present invention, in the above method, the reaction is performed in an organic solvent, wherein the organic solvent used is selected from formamide, chloroform, DMF, acetonitrile, tetrahydrofuran, N-methylpyrrolidone and the like, preferably N-methylpyrrolidone.

According to an embodiment of the present invention, in the above method, the reaction is performed in the presence of an alkaline compound selected from anhydrous Na₂CO₃, anhydrous K₂CO₃, NaHCO₃, Cs₂CO₃ and the like, preferably Cs₂CO₃.

According to an embodiment of the present invention, in the above method, the reaction temperature is 30-80° C., preferably 50-55° C.; the heating time is 12-36 h, preferably 18-24 h.

According to an embodiment of the present invention, different compounds are obtained by the above reaction according to different feeding molar ratios, and when the molar ratio of the compound of formula (II) to the functional groups of R₁-L is 1:1.1-1:1.5, a compound with all R not being H can be obtained; when the molar ratio of the compound (II) to the functional groups of R₁-L is 1:0.5-1:1, a compound with part of R not being H can be obtained.

According to an embodiment of the present invention, the reaction product of the above reaction can be diluted with dichloromethane, chloroform, ethyl acetate and the like, preferably chloroform.

According to an embodiment of the present invention, when R in the compound of formula (I) is

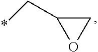 [SMILES: *CC1CO1]

the synthesis method comprises: reacting a compound of formula (II) with epibromhydrin in an organic solvent in the presence of an alkaline compound under stirring and heating according to different feeding molar ratios, diluting the resulting mixture with an organic solvent after the reaction is completed, washing the solution with deionized water, drying the solution with anhydrous Na₂CO₃, and subjecting to solvent exchange with methanol to give the compound of formula (I).

The present invention further provides use of the compound of formula (I) as described above, used as a multifunctional cross-linking agent or used in a photoresist composition.

The present invention further provides a negative photoresist composition comprising the compound of formula (I) as described above.

According to an embodiment of the present invention, when R in the compound of formula (I) is allyl, the allyl can be oxidized to an alkylene oxide using a conventional method.

According to an embodiment of the present invention, the negative photoresist composition further comprises a photoacid generator, a photoresist solvent, other additives and the like.

According to an embodiment of the present invention, the negative photoresist composition comprises 2.5-10% of photoacid generator (the mass ratio is relative to the compound of formula (I)).

According to an embodiment of the present invention, the photoacid generator comprises ionic or non-ionic acid generators such as triphenylsulfonium triflate, triphenylsulfonium nonaflate, bis(4-tert-butylphenyl)iodonium p-toluenesulfonate and N-hydroxynaphthalimide triflate, and is preferably N-hydroxynaphthalimide triflate.

According to an embodiment of the present invention, the photoresist solvent comprises propylene glycol monomethyl ether acetate (PGMEA), ethyl lactate, ethylene glycol monomethyl ether, cyclohexanone and the like, and is preferably propylene glycol monomethyl ether acetate (PGMEA).

According to an embodiment of the present invention, the negative photoresist composition may further comprise other additives, such as surfactants and stabilizers.

The compound of formula (I) in the negative photoresist composition provided herein has relatively high thermal stability, does not precipitate during baking and is not easily denatured during photolithography. The present invention further provides use of the negative photoresist composition as described above in electron-beam lithography, UV lithography (365 nm), DUV lithography (248 nm, 193 nm) and EUVL (13.5 nm).

### Beneficial Effects

The compounds of formula (I) described herein are obtained by introducing an epoxy group or an allyl group oxidizable to form an epoxy group on a compound having a bisphenol A skeleton structure. In the exposure process, the epoxy groups are ring-opened to form a cross-linked network. This film is a highly cross-linked film, which has excellent mechanical strength and can improve the pattern collapse as compared with a non-cross-linked film. There is little or no gas produced in the exposure process. Meanwhile, as the epoxy group can form oxonium, the cationic active site is linked with the growing cross-linked network, and thus the control of the diffusion of the acid generator can be realized. This is because that, on one hand, the cross-linked network formed by the ring-opening of the epoxy can wrap the acid generator; on the other hand, the acid generator is attached onto the epoxy ring.

The compounds of formula (I) in the negative photoresist compositions provided herein have relatively high thermal stability, do not precipitate during baking and are not easily denatured during photolithography. The negative molecular glass photoresists have good film-forming property, high thermal stability, less proneness to properties varying during storage, and low viscosity, no need for additional solvents for dilution during use. After exposure, the exposed pattern shows high contrast, excellent resolution and good sensitivity, and can present the lithographic line width of 3.5 μm.

The compounds provided herein feature simple molecular structure, controllable molecular weight, simple synthesis steps.

### BRIEF DESCRIPTION OF THE DRAWINGS

FIG. 1 is a thermogravimetric analysis diagram of the epoxy compound (I-a) in Example 1; and

FIG. 2 is SEM diagrams of the epoxy compound (I-a) in Example 1 exposed at 365 nm (UV), wherein (a) is the exposure dose and line width; (b) is a dense line graph; (c) is a rectangular dot matrix graph; (d) is a circular dot matrix graph; (e) is an L-shaped graph; and (f) is a concentric circular graph.

### DETAILED DESCRIPTION

The technical solutions of the present invention will be further illustrated in detail with reference to the following specific examples. It should be understood that the following examples are merely exemplary illustration and explanation of the present invention, and should not be construed as limiting the protection scope of the present invention. All techniques implemented based on the aforementioned contents of the present invention are encompassed within the protection scope of the present invention.

Unless otherwise specified, the raw materials and reagents used in the following examples are all commercially available products or can be prepared by known methods.

BPA-6OH is prepared in the following examples with reference to the method in Patent No. ZL201210156675.6.

### EXAMPLE 1. SYNTHESIS OF EPOXY COMPOUND (I-a)

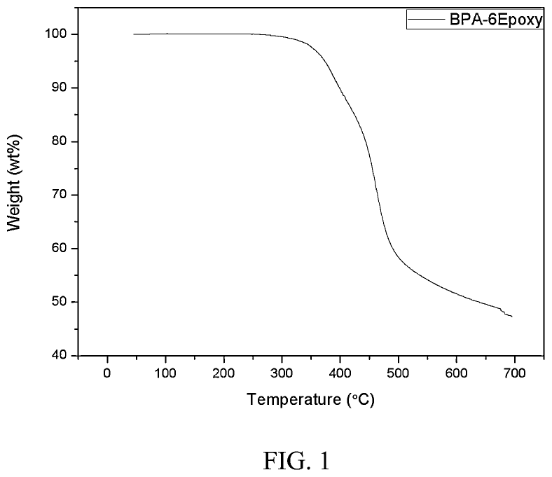

5.98 g (10 mmol) of BPA-60H and 21.50 g (66 mmol) of Cs2CO3 were added into a 150 mL three-necked flask sequentially, followed by the addition of 7 mL (70 mmol) of epibromhydrin and 15 mL of N-methylpyrrolidone, and the mixture was stirred at 50-55° C. under reflux for 18-24 h. After the reaction was completed, the reaction mixture was diluted with chloroform, washed three times with deionized water, dried with anhydrous Na₂CO₃ under stirring, then subjected to solvent exchange with methanol, and dried at 60° C. under vacuum to give the epoxy compound (I-a) completely protected with epoxy groups (5.05 g, 54% yield) in the form of a pale yellow solid. MALDI-TOF (C₅₇H₅₆O₁₂), m/z: 932.376. The TGA diagram is shown in FIG. 1, showing that the thermal stability is high, and only 5% of the mass is lost at around 370° C.

The structure of the raw material BPA-60H is as follows:

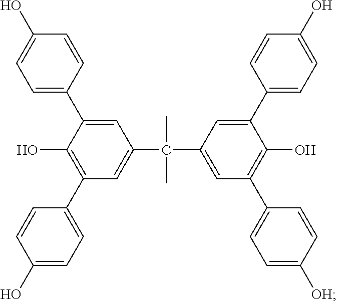 [SMILES: CC(C)(c1cc(-c2ccc(O)cc2)c(O)c(-c2ccc(O)cc2)c1)c1cc(-c2ccc(O)cc2)c(O)c(-c2ccc(O)cc2)c1]

the structure of the product epoxy compound (I-a) is as follows:

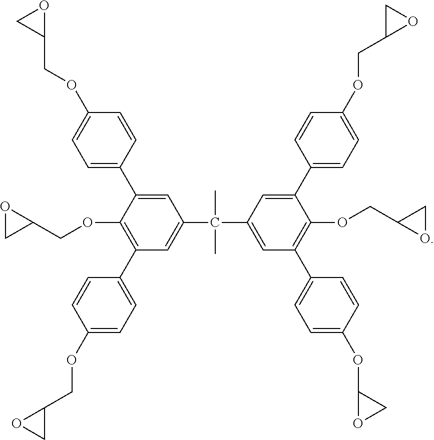 [SMILES: CC(C)(c1cc(-c2ccc(OCC3CO3)cc2)c(OCC2CO2)c(-c2ccc(OCC3CO3)cc2)c1)c1cc(-c2ccc(OCC3CO3)cc2)c(OCC2CO2)c(-c2ccc(OC3CO3)cc2)c1]

### EXAMPLE 2. PREPARATION OF NEGATIVE PHOTORESIST COMPOSITION COMPRISING EPOXY COMPOUND (I-a)

300 mg of the epoxy compound (I-a) completely protected with epoxy groups prepared in Example 1, 22.5 mg of N-hydroxynaphthalimide triflate as a photoacid generator and 10 mL of propylene glycol monomethyl ether acetate (PGMEA) as a photoresist solvent were mixed to give a photoresist solution, and the solution was filtered three times with a 0.20 μm polytetrafluoroethylene film after half an hour of ultrasonic treatment to give a negative photoresist composition.

### EXAMPLE 3. PHOTOLITHOGRAPHY PERFORMANCE OF NEGATIVE PHOTORESIST COMPOSITION COMPRISING EPOXY COMPOUND (I-a)

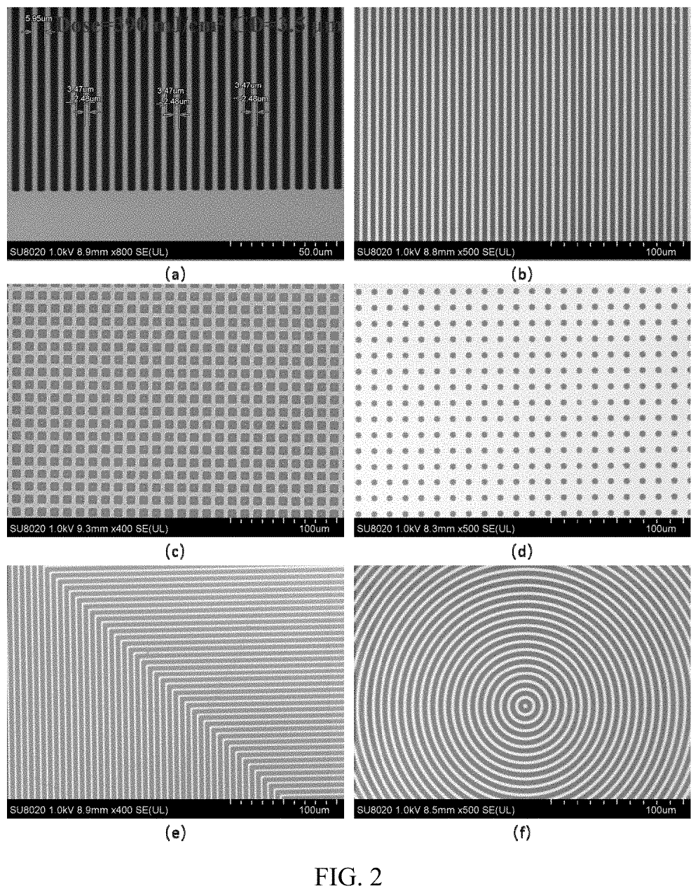

30 mg/mL negative photoresist composition was prepared according to Example 2, an untreated blank silicon wafer was selected, the spin-coating parameter was set to 3000 rpm/90 s, the pre-baking parameter was set to 80° C./120 s, and the film thickness was about 50 nm as measured by an ellipsometer. The UV lithography (365 nm) was performed using a front-aligned UV lithography machine from ABM-USA, Inc, with an exposure time set to 30 s, a post-baking parameter of 90° C./120 s, a development parameter of methyl isobutyl ketone/30 s, and a rinsing parameter of isopropanol/30 s. SEM diagrams are acquired using Hitachi 8020 scanning electron microscope after exposure, and specific photolithography results are shown in FIG. 2. As can be seen from FIG. 2, the resulting photoresist composition has high resolution, excellent sensitivity and high contrast.

The examples of the present invention have been described above. However, the present invention is not limited to the above examples. Any modification, equivalent, improvement and the like made without departing from the spirit and principle of the present invention shall fall within the protection scope of the present invention.

1. A compound of formula (I): 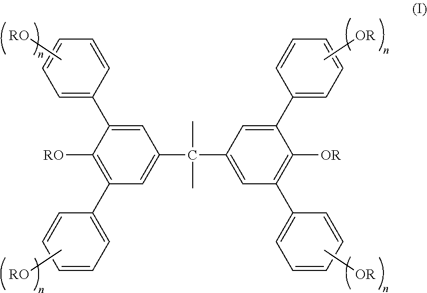
wherein R is H, 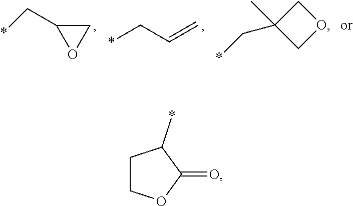
provided that R is not all H; the * in the above groups is a linking site; n is 1, 2, 3, 4 or 5.

2. The compound according to claim 1, wherein the compound of formula (I) is selected from a compound of formula (IA) and a compound of formula (IB) below,

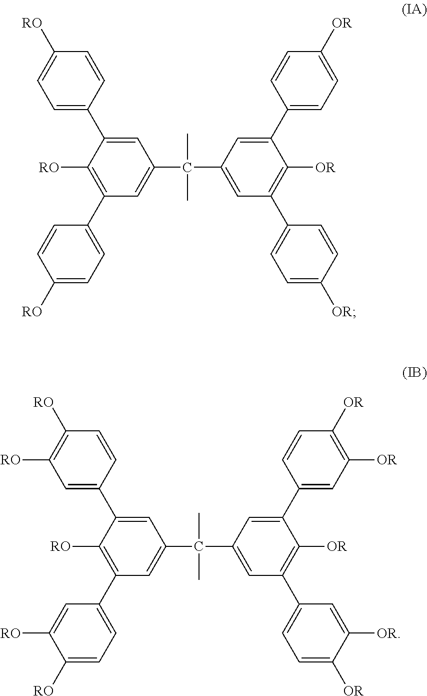

3. The compound according to claim 1, wherein R is

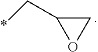 [SMILES: *CC1CO1]

4. The compound according to claim 1, wherein the compound of formula (I) is selected from the following compounds:

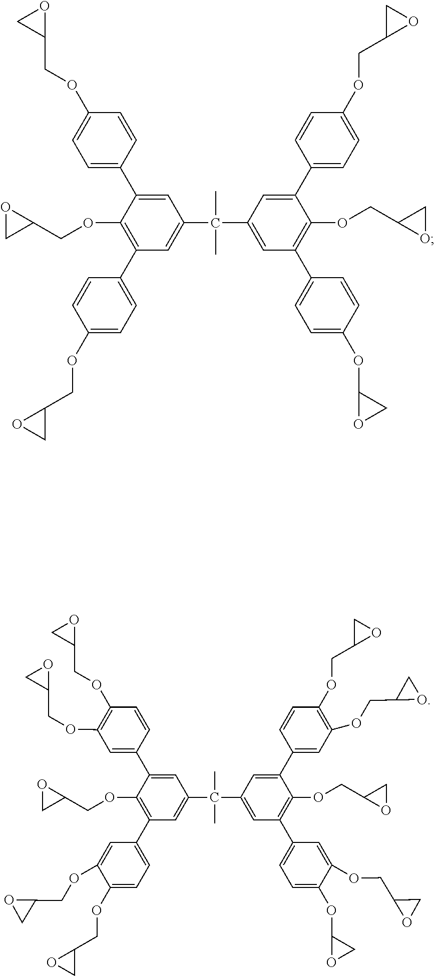 [SMILES: CC(C)(c1cc(-c2ccc(OCC3CO3)cc2)c(OCC2CO2)c(-c2ccc(OCC3CO3)cc2)c1)c1cc(-c2ccc(OCC3CO3)cc2)c(OCC2CO2)c(-c2ccc(OC3CO3)cc2)c1 ; CC(C)(c1cc(-c2ccc(OCC3CO3)c(OCC3CO3)c2)c(OCC2CO2)c(-c2ccc(OCC3CO3)c(OCC3CO3)c2)c1)c1cc(-c2ccc(OCC3CO3)c(OCC3CO3)c2)c(OCC2CO2)c(-c2ccc(OC3CO3)c(OCC3CO3)c2)c1]

5. A method for synthesizing the compound of formula (I) according to claim 1, comprising: reacting a compound of formula (II) with R₁-L to give the compound of formula (I); 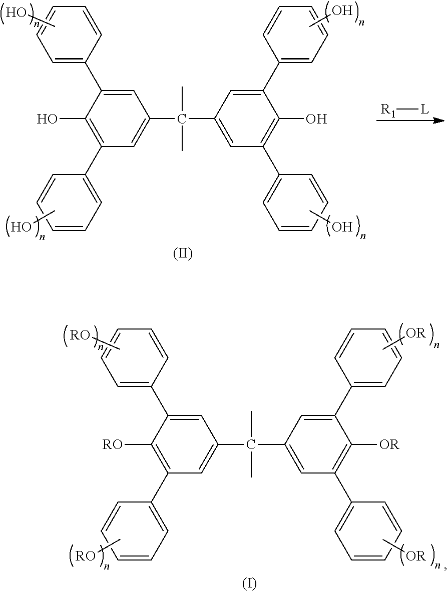
wherein R and n are as defined in claim 1, R₁ is 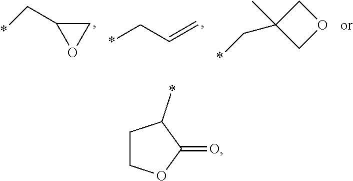
and L is selected from a leaving group such as halogen or p-toluenesulfonate.

6. The method according to claim 5, wherein R₁-L is selected from epibromhydrin, allyl bromide, α-bromo-γ-butyrolactone and 3-methyl-3-(tosyloxymethyl)oxetane.

7. Use of the compound of formula (I) according to claim 1 as a multi-functional cross-linking agent or in a photoresist composition.

8. A negative photoresist composition, comprising the compound of formula (I) according to claim 1.

9. The negative photoresist composition according to claim 8, further comprising a photoacid generator, a photoresist solvent and other additives;
preferably, the negative photoresist composition comprises 2.5-10% (mass ratio relative to that of the compound of formula (I)) of photoacid generator;
preferably, the photoacid generator comprises ionic or non-ionic acid generators, such as triphenylsulfonium triflate, triphenylsulfonium nonaflate, bis(4-tert-butylphenyl)iodonium p-toluenesulfonate and N-hydroxynaphthalimide triflate;
preferably, the photoresist solvent comprises propylene glycol monomethyl ether acetate (PGMEA), ethyl lactate, ethylene glycol monomethyl ether and cyclohexanone.

10. Use of the negative photoresist composition according to claim 8 in electron-beam lithography, ultraviolet lithography, deep ultraviolet lithography and extreme ultraviolet lithography.
